(19) United States
(12) Patent Application Publication
(10) Pub. No.: US 2026/0196311 A1
(43) Pub. Date: Jul. 9, 2026

(54) DISCRETE DIFFUSION MODEL FOR GENERALIST DRUG DISCOVERY

(71) Applicant: NVIDIA Corp., Santa Clara, CA (US)

(72) Inventors: Weili Nie, Sunnyvale, CA (US); Seul Lee, Seoul (KR); Karsten Julian Kreis, Vancouver (CA); Srimukh Prasad Veccham Krishna Prasad, San Carlos, CA (US); Meng Liu, San Jose, CA (US); Daniel Alexander Reidenbach, San Jose, CA (US); Saee Paliwal, Brooklyn, NY (US); Arash Vahdat, San Mateo, CA (US)

(73) Assignee: NVIDIA Corp., Santa Clara, CA (US)

(21) Appl. No.: 19/443,271

(22) Filed: Jan. 8, 2026

Related U.S. Application Data
(60) Provisional application No. 63/743,162, filed on Jan. 8, 2025.

(51) Int. Cl. G16C 20/50; G16C 20/70

(52) U.S. Cl. CPC G16C 20/50; G16C 20/70

(57) ABSTRACT

Candidate drug exploration processes utilizing a masked discrete diffusion model with fragment remasking to generate molecular sequences, wherein the operation of the masked discrete diffusion model is conditioned with molecular context guidance, and fragment remasking involves generating molecular fragment candidates, masking the molecular fragment candidates, scoring the molecular fragment candidates, and attaching the molecular fragment candidates to a molecule.

### CROSS-REFERENCE TO RELATED APPLICATIONS

This application claims priority and benefit under 35 U.S.C. 119 (e) to U.S. Application Ser. No. 63/743,162, “GenMol: A Drug Discovery Generalist with Discrete Diffusion”, filed on Jan. 8, 2025, the contents of which are incorporated herein by reference in their entirety.

### BACKGROUND

Autoregressive generative artificial intelligence models generate outputs in a sequential manner, where each current output is conditioned on previous outputs. These models operate by modeling the probability distribution of the output sequence. An autoregressive model predicts the next element in a sequence based on past elements. For example, in text generation, a model like the Generative Pre-trained Transformer (GPT) generates the next word considering all preceding words.

An autoregressive model estimates the probability of possible subsequent elements. It uses mechanisms such as neural networks to compute these probabilities. Once the probabilities are determined, the model samples from these probabilities to decide the next output. This process can continue iteratively to create a complete sequence. An autoregressive model may be trained on large datasets using maximum likelihood estimation to fine-tune parameters so that the predicted probabilities closely match the training data distribution.

Autoregressive models can capture complex dependencies in temporal or sequential data, making them well-suited for applications in text synthesis, speech generation, and more.

Discrete diffusion generative artificial intelligence models are a class of generative models that simulate the process of diffusion, which gradually converts random noise into structured data through a series of reversible transformations. These models work by applying a forward diffusion process to data, where data points are progressively perturbed by random noise through a series of discrete time steps, leading them towards a noise-like prior distribution. The model then learns a reverse process to revert the perturbed data back to its original form.

The process involves a forward diffusion process, during which the original (training) data is subjected to incremental noise addition over multiple steps, transitioning it gradually to a distribution that resembles noise. The model is trained through a neural network to learn the reverse of the forward process, effectively denoising the data step by step, moving it back from noise to its original structured form.

A discrete diffusion model may be trained using a variational inference approach, optimizing a modified version of the variational lower bound that is tailored for the diffusion process. The loss function often involves terms that guide the model in accurately predicting the noise added at each step and reconstructing the data. Discrete diffusion models are used in scenarios where data models require a discrete state space, such as images or categorical data.

Training a masked discrete diffusion model begins with a training dataset comprising discrete variables. This can include text sequences, categorical data, or any type of data that can be represented in discrete form.

A masking process is applied to the training data. This involves hiding or altering parts of the input data to create a form of noise. The masking aims to simulate incomplete versions of the data for training and is similar to techniques used in masked language models.

A diffusion process is configured over the discrete states. This involves specifying the forward process, where data transitions from the original form to a fully noised state through a series of discrete steps. A neural network is trained to learn the reverse diffusion process. The goal is to recover the original data from the noisy version through these learned transformations. Typically, models like transformers or recurrent neural networks may be employed given their capacity to handle sequential, discrete data.

An appropriate loss function is configured that facilitates learning of the reverse process. This often involves comparing the predicted clean data with the original data, utilizing metrics suitable for discrete variables. Optimization techniques are also applied to train the model, adjusting parameters to minimize the loss. Stochastic gradient descent or its variants are commonly used for this purpose.

The process iteratively trains the model to become adept at denoising the masked, noisy data, enabling it to effectively perform tasks like prediction, completion, or generation of discrete data sequences.

Generative models have emerged as a promising methodology to accelerate labor-intensive drug discovery pipelines, but conventional molecular generative models tend to focus on only one or two of the drug discovery scenarios. They either cannot be applied to multiple tasks or require expensive modifications including retraining of a specific architecture for each task.

SAFE-GPT uses Sequential Attachment-based Fragment Embedding (SAFE) of molecules, which represents a molecule as an unordered sequence of Simplified Molecular Input Line Entry System (SMILES) fragment blocks. However, since all sequence-based molecular representations including SMILES and SAFE assume an ordering of tokens based on heuristic rules such as depth-first search (DFS), autoregressive models that operate in a left-to-right order like GPT are unnatural for processing and generating molecular sequences. In addition, the autoregressive decoding scheme limits the computational efficiency of the model and makes it challenging to introduce guidance during generation. Moreover, SAFE-GPT relies on finetuning under expensive reinforcement learning (RL) objectives to be applied to goal-directed molecule generation.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 depicts an example of the macro structure of a BERT network.

FIG. 2 depicts a goal-directed molecular optimization algorithm in accordance with one embodiment.

FIG. 3 depicts a molecular structure generative model and process in one embodiment.

FIG. 4 depicts a goal-directed hit generation and lead optimization process in one embodiment.

FIG. 5 depicts a parallel processing unit in accordance with one embodiment.

FIG. 6 depicts a general processing cluster in accordance with one embodiment.

FIG. 7 depicts a memory partition unit in accordance with one embodiment.

FIG. 8 depicts a streaming multiprocessor in accordance with one embodiment.

FIG. 9 depicts a processing system in accordance with one embodiment.

FIG. 10 depicts an exemplary processing system in accordance with another embodiment.

### DETAILED DESCRIPTION

Disclosed herein are mechanisms comprising a single discrete diffusion model applicable to diverse drug discovery scenarios. The disclosed mechanisms generate Sequential Attachment-based Fragment Embedding (SAFE) sequences through non-autoregressive bidirectional parallel decoding, thereby enabling the utilization of a molecular context that does not rely on the specific token ordering with improved sampling efficiency over conventional mechanisms.

The disclosed mechanisms apply fragments as basic building blocks for molecules and implement fragment remasking, a process that optimizes molecules by regenerating masked fragments, enabling effective exploration of chemical space. The disclosed mechanisms may further implement molecular context guidance (MCG), a process tailored for masked discrete diffusion.

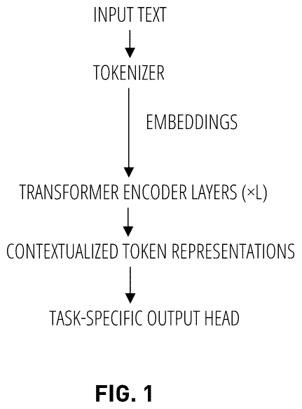

The disclosed mechanisms implement a masked discrete diffusion framework with a BERT model structure to generate SAFE molecular sequences. The macro structure of a BERT network comprises a deep stack of Transformer encoders that learns bidirectional, context-aware representations of sequences (e.g., text) by jointly attending to all tokens in the sequence (FIG. 1).

The utilization of discrete diffusion enables the disclosed mechanisms to exploit a molecular context that does not rely on the specific ordering of tokens and fragments by bidirectional attention, and further enables exploration of a chemical space via a remasking mechanism. The utilization of non-autoregressive parallel decoding improves computational efficiency.

Fragment remasking as defined herein comprises a molecular optimization process that replaces certain fragments in a given molecule with masked tokens, from which diffusion generates new fragments. Discrete diffusion enables the application of guidance during generation based on an entire sequence. Molecular context guidance may be utilized to improve the performance of the discrete diffusion model by calibrating its predictions with information in a given molecular context.

Fragment-based molecular generative models refers to a class of models that reassemble existing molecular substructures (i.e., fragments) to generate new molecules. Assembling fragments simplifies the generation process and improves chemical validity. The unit that determines biochemical effect of a molecule is a fragment rather than an individual atom.

Define x as a sequence of £ tokens, each of which, denoted as x, is a one-hot vector with K categories

$$( i . e . , {x}_{i}^{l} ∈ {\{ 0 , 1 \}}^{K} and {\sum }_{i = 1}^{K} {x}_{i}^{l} = 1 ) .$$

Without loss of generality, assume the K^{th} category represents the masking token, whose one-hot vector is denoted by m (i.e., m_{K}=1). Define Cat (·; π) as a categorical distribution with a probability π∈Δ^{K}, where Δ^{K} represents the simplex over K categories.

A forward masking process independently interpolates the probability mass between each token in clean data sequence x^{l} and the masking token m, defined as

$$q ( {z}_{t}^{l} \vert {x}^{1} ) = C a t ( {z}_{t}^{l} ; {α}_{t} {x}^{1} + ( 1 - {α}_{t} ) m ) \qquad Eq . 1$$

where

$${z}_{t}^{l}$$

denotes the l^{th} token in the noisy data sequence at the time step t∈[0,1], and αₜ∈[0,1] denotes a masking ratio that is monotonically decreasing function of t, with α₀=1 to α₁=0. Accordingly, at time step t=1, zₜ becomes a sequence of all masked tokens.

The reverse unmasking process inverts the masking process and independently infers each token of the less masked data zₛ from more masked data zₜ, with s<t, which is given by

$$p θ ( {z}_{s}^{l} \vert {z}_{t}^{l} ) = \{ \begin{matrix}Cat ( {z}_{s}^{l} ; {z}_{t}^{l} ) & {z}_{t}^{l} \neq m \\ Cat ( {z}_{s}^{l} ; \frac{( 1 - {α}_{s} ) m + ( {α}_{s} - {α}_{t} ) {x}_{θ}^{l} ( {z}_{t} , t )}{1 - {α}_{t}} ) & {z}_{t}^{l} = m\end{matrix} \qquad Eq . 2$$

where x_{θ}(zₜ, t) is a denoising network that takes the noisy data sequence zₜ as input and predicts L probability vectors for the clean data sequence. This parameterization designs the reverse process such that it does not change unmasked tokens. To train the denoising network x_{θ}(zₜ, t), the training objective may be set to:

$${L}_{NELBO} = {𝔼}_{q} {\int }_{0}^{1} \frac{{α}_{t}^{l}}{1 - {α}_{t}} {\sum }_{l} log ( {x}_{θ}^{l} ( {z}_{t} , t ) , {x}^{l} ) d t \qquad Eq . 3$$

which is the weighted average of MLM losses (i.e., cross-entropy losses) over time steps. MLM loss stands for Masked Language Modeling loss, a core training objective for BERT-type language models (and other bidirectional Transformer encoders). Applying MLM loss configures the model to understand context in both directions—by predicting deliberately hidden (masked) words in a sentence based on the surrounding words.

NELBO refers for Negative Evidence Lower Bound, a loss function used in training variational or generative AI models, such as Variational Autoencoders (VAEs), and some diffusion or latent-variable models. NELBO measures how far the model's learned probability distribution is from the true data distribution, balancing the accurate reconstruction of observed data with maintaining the latent representation smooth and regularized (so that it generalizes well).

SAFE represents molecules as an unordered sequence of fragment blocks, thereby casting molecular design into sequence completion. SAFE is a non-canonical SMILES in which the arrangement of SMILES tokens corresponding to the same molecular fragment is consecutive.

Molecules are decomposed into fragments by the BRICS algorithm (Breaking of Retrosynthetically Interesting Chemical Substructures) and the fragments are concatenated using, for example, a dot token (“.”) while preserving their attachment points. SAFE is permutation-invariant on fragments, i.e., the order of fragments within a SAFE string does not change the molecular identity.

The disclosed mechanisms utilize masked discrete diffusion to generate SAFE sequences and establish a flexible and efficient molecule generation framework. A BERT model architecture may be utilized for the denoising network xe along with the training scheme of masked diffusion language models (MDLM, e.g., Sahoo, S. S. et al, “Simple and effective masked diffusion language model”, Advances in Neural Information Processing Systems, 2024).

MDLMs utilize a discrete diffusion/denoising process over token sequences. By combining masked-language modeling (MLM) concepts with a diffusion/denoising framework, MDLMs enable bidirectional context understanding (like BERT-style models) plus generative capability (sequence generation)—something classical MLM models lack.

MDLM defines a forward corruption process. Starting from a clean token sequence, it gradually “masks out” tokens (replace with a [MASK] token) according to a noise schedule. This progressively increases the fraction of masked tokens as the time step advances. At time step 0 clean data is applied; at final time T, the applied sequence is mostly masked. The training objective then is for a neural network (“denoiser”) to reverse this process—reconstructing the original sequence from the corrupted one. The training objective may comprise a simplified variational lower bound (ELBO) objective—more specifically a Rao-Blackwellized ELBO—which under discrete masking becomes a mixture of classical masked-language modeling cross-entropy losses. The loss is conceptually akin to “predict the masked tokens,” but framed within a diffusion/probabilistic denoising framework.

MDLM models typically implement encoder-only transformer backbone—similar to BERT—rather than a causal decoder. Because it's not inherently autoregressive, MDLM can in principle consider bidirectional context when denoising/predicting tokens. To generate a sequence, MDLM starts from a fully masked sequence (or a sequence with a high fraction of masks), then iteratively “unmasks” (denoises) tokens over several steps according to its learned reverse diffusion process.

At each masked index l, the disclosed mechanisms sample

$${z}_{s}^{l}$$

based on the reverse unmasking process

$${p}_{θ}^{l} := {p}_{θ} ( {z}_{s}^{l} \vert {z}_{t} )$$

specified by:

$${p}_{θ , i}^{l} = \frac{exp ( log {x}_{θ , i}^{l} ( {z}_{t} , t ) / τ}{{\sum }_{j = 1}^{K} exp ( log {x}_{θ , j}^{l} ( {z}_{t} , t ) / τ} for i = 1 , … , K , \qquad Eq . 4$$

where τ is the Softmax temperature. All masked tokens are predicted in a parallel manner and the top N confident predictions are confirmed with additional randomness r, where N is the number of tokens to unmask at each time step. Trade-offs between molecular quality and diversity often arise in drug discovery. These may be balanced by configuring parameters for Softmax temperature τ and the randomness r.

Goal-directed molecular optimization tasks may implement a generation process comprising: fragment candidate generation, fragment candidate scoring, fragment attaching, and fragment remasking.

A set of D molecules

$${D = [ ( {x}_{d} , y ( {x}_{d} ) ) \}}_{d = 1}^{D} ,$$

where y(x_{d}) is the target property of molecule xⱼ, may be decomposed into a set of F fragments

$$F = {\{ {f}_{k} \}}_{K = 1}^{F}$$

using a configured decomposition rule. The score of a given fragment fₖ may be determined as:

$$y ( {f}_{k} ) = \frac{1}{\vert S ( {f}_{k} ) \vert} {\sum }_{x ∈ S ( {f}_{k )}} y ( x ) \qquad Eq . 5$$

where S(fₖ)={x: fₖ is a subgraph of x} and the top V fragments based on score are selected as a vocabulary.

During generation, a molecule xᵢₙᵢₜ is first generated by randomly selecting two fragments from the vocabulary and attaching them. Fragments or functional groups influence the chemical properties of a molecule and therefore fragments that commonly occur in molecules with desirable properties are likely to carry those properties over to new molecules. However, with fragment attaching alone, the model cannot generate new fragments that are not included in the initial vocabulary, resulting in suboptimal exploration in chemical space.

It may be suboptimal to naively adopt the same strategy for small molecule optimization as used conventionally for large molecules such as protein sequences. This is because each token in a SAFE (or SMILES) sequence represents a single atom or bond, and masking them individually results in localized and ineffective exploration.

Discrete diffusion with fragment remasking explores a neighborhood of a given molecule in chemical space to find optimal molecules. Discrete diffusion enables masking and re-predicting some tokens of a given molecule. Fragment remasking modifies a molecule by randomly re-predicting a fragment. Given xᵢₙᵢₜ>xₘₐₛₖ is constructed by randomly selecting one of the fragments of xᵢₙᵢₜ and replacing it with a mask chunk. New fragment x_{new} is generated by iteratively unmasking xₘₐₛₖ using discrete diffusion. A finer decomposition rule than the one that constructed the vocabulary may be applied at this stage, enabling fragment re-masking to operate at a fine-grained level.

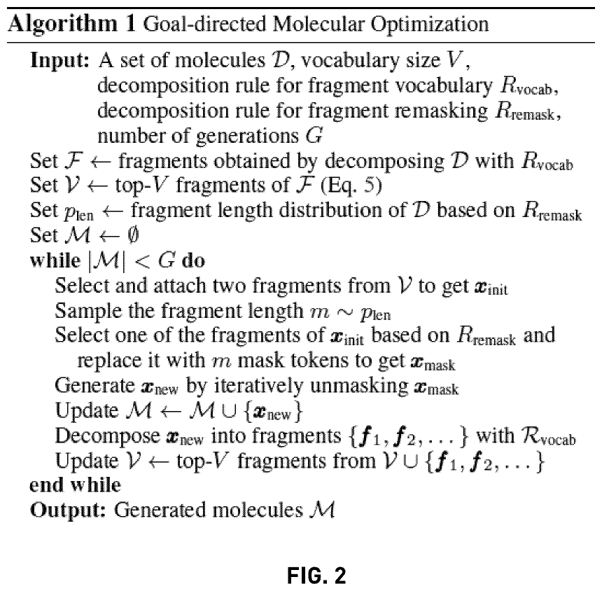

Fragment remasking may be implemented by decomposing and scoring a fragment x_{new} by Eq. (5), with the fragment vocabulary dynamically updated by selecting the top V fragments, enabling exploration beyond the initial fragments. Algorithm 1 (FIG. 2) depicts logic for this implementation.

Instead of using a fixed length, the lengths of the fragment mask chunks may be sampled from a predefined distribution, e.g., the distribution of fragment lengths in the training set. The length of the fragment depends on the decomposition rule used, enabling the automatically adjustment of fragment length based on the rule. This also ensures the generation of fragments of varying lengths, increasing the controllability of molecule generation.

Assuming a SAFE molecular sequence x is comprised of F fragments, x may be represented as a set of the fragments

$${\{ {f}_{k} \}}_{k = 1}^{F} ,$$

where fₖ denotes an attachment point-assigned SAFE fragment. To sample a molecule x from p(x)=p(f₁ . . . , f_{F}), fragment re-masking repeats the process of uniformly selecting the index k and then sampling fₖ from p(fₖ|f_{\k}) where f_{\k} denotes f₁, . . . f_{F} but with fₖ omitted. Fragment remasking may thus be implemented in some embodiments as Gibbs sampling with the Markov kernel p(fₖ|f_{\k}), enabling a random walk in the neighborhood of the given molecule x.

Molecular context guidance (MCG) comprises a process tailored for masked discrete diffusion by the disclosed mechanisms. Generalized, classifier-free guidance (CFG) may be utilized to extrapolate between the predictions of two denoising networks:

$${D}^{( w )} ( {z}_{s} \vert {z}_{t} , y ) = w {D}_{1} ( {z}_{s} \vert {z}_{t} , y ) + ( 1 - w ) {D}_{0} ( {z}_{s} \vert {z}_{t} , y ) \qquad Eq . 6$$

where D₁ and D₀ are a high-quality model and a poor model, respectively, y is the condition to perform the guidance on, and w>1 is the guidance scale. The same network D_{θ} may be utilized for D₁ and D₀ with additional degradations applied to D₀, such as corrupted input, e.g., CFG sets D₁=D_{θ}(zₛ|zₜ, y) and D₀=D_{θ}(zₛ|zₜ, Ø). With autoguidance the weaker version of the model amplifies the errors. Emphasizing the output of D₁ over D₀ by setting w>1 eliminates or reduces these errors.

The disclosed models may be trained under an MDLM framework with autoguidance for the MDLM formulation. Specifically, D₁ may be configured as the original prediction of the denoiser x_{θ} and D₀ as the prediction with partially corrupted input, replacing the logits in Eq. 4 by

$$log {x}_{θ , i}^{( w ) , l} ( {z}_{t} , t ) := w log {x}_{θ , i}^{l} ( {z}_{t} , t ) + ( 1 - w ) log {x}_{θ , i}^{l} ( {\tilde{z}}_{t} , t ) \qquad Eq . 7$$

where \tilde{z}ₜ is constructed by masking γ·100% of the tokens in zₜ. Eq. (7) compares different behaviors of the generator model given the original and masked molecular contexts and calibrate its predictions based on this difference. Using this approach, the disclosed mechanisms may fully utilize the given molecular context information in fragment-constrained and goal-directed generation.

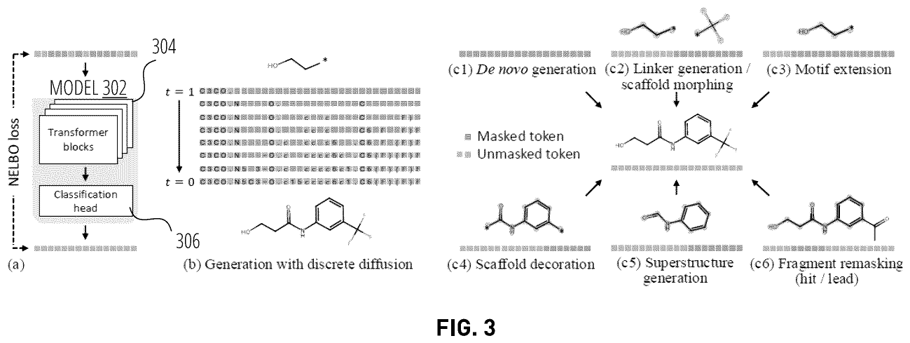

FIG. 3 depicts a molecular structure generative model 302 and process in one embodiment. The generative model 302 comprises a BERT structure of transformer blocks 304 and a classifier network 306 trained with a NELBO loss of masked discrete diffusion. At (b) using masked discrete diffusion, the generative model completes a molecule by simulating backward in time and predicting masked tokens at each time step t until all tokens are unmasked. At (c), various molecular discovery tasks enabled by the generative model are depicted. (c1) depicts de novo molecule generation, (c2-c5) depict fragment-constrained generation, and (c6) depicts fragment remasking be applied to goal-directed hit generation and lead optimization.

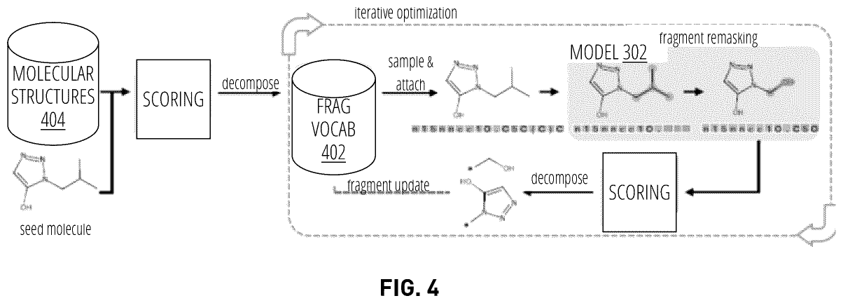

FIG. 4 depicts a goal-directed hit generation and lead optimization process in one embodiment. An initial fragment vocabulary 402 is constructed by scoring and decomposing existing molecular structures 404 (hit generation) or a seed molecule (lead optimization). Two fragments are randomly sampled from the fragment vocabulary 402 and attached, and the generative model 302 is operated to perform fragment remasking. The fragment vocabulary is updated with the generated molecules for the next iteration. With fragment remasking, the generative model may explore beyond the initial fragment vocabulary to find chemical optima.

The mechanisms disclosed herein may be implemented in and/or by computing devices utilizing one or more graphic processing unit (GPU, e.g., parallel processing unit 502, parallel processing module 902) and/or general purpose data processor (e.g., a “central processing unit” or CPU, such as central processing unit 904). A non-volatile machine-readable media, e.g., memory 504, main memory 1002) may be configured with instructions that when applied to the GPU/CPU configure the computer system to implement the disclosed mechanisms. A graphics processing unit may be a standalone chip or package, or may comprise graphics processing circuitry integrated with a central processing unit. Exemplary architectures will now be described that may be configured to implement the mechanisms disclosed herein.

The following description may use certain acronyms and abbreviations as follows:
“DPC” refers to a “data processing cluster”;
“GPC” refers to a “general processing cluster”;
“I/O” refers to a “input/output”;
“L1 cache” refers to “level one cache”;
“L2 cache” refers to “level two cache”;
“LSU” refers to a “load/store unit”;
“MMU” refers to a “memory management unit”;
“MPC” refers to an “M-pipe controller”;
“PPU” refers to a “parallel processing unit”;
“PROP” refers to a “pre-raster operations unit”;
“ROP” refers to a “raster operations”;
“SFU” refers to a “special function unit”;
“SM” refers to a “streaming multiprocessor”;
“Viewport SCC” refers to “viewport scale, cull, and clip”;
“WDX” refers to a “work distribution crossbar”; and
“XBar” refers to a “crossbar”.

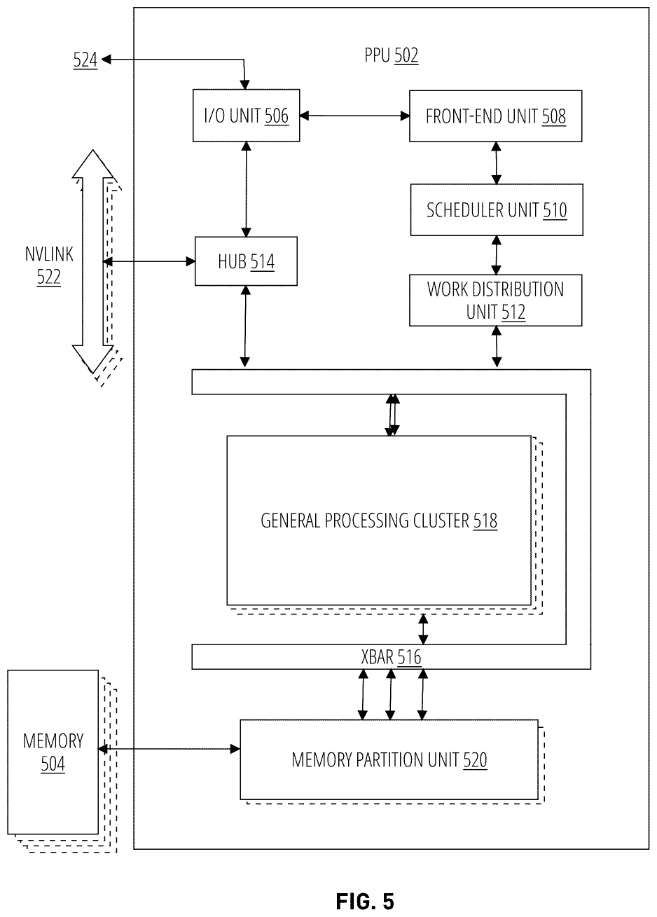

FIG. 5 depicts a parallel processing unit 502, in accordance with an embodiment. In an embodiment, the parallel processing unit 502 is a multi-threaded processor that is implemented on one or more integrated circuit devices. The parallel processing unit 502 is a latency hiding architecture designed to process many threads in parallel. A thread (e.g., a thread of execution) is an instantiation of a set of instructions configured to be executed by the parallel processing unit 502. In an embodiment, the parallel processing unit 502 is a graphics processing unit (GPU) configured to implement a graphics rendering pipeline for processing three-dimensional (3D) graphics data in order to generate two-dimensional (2D) image data for display on a display device such as a liquid crystal display (LCD) device. In other embodiments, the parallel processing unit 502 may be utilized for performing general-purpose computations. While one exemplary parallel processor is provided herein for illustrative purposes, it should be strongly noted that such processor is set forth for illustrative purposes only, and that any processor may be employed to supplement and/or substitute for the same.

One or more parallel processing unit 502 modules may be configured to accelerate thousands of High Performance Computing (HPC), data center, and machine learning applications. The parallel processing unit 502 may be configured to accelerate numerous deep learning systems and applications including autonomous vehicle platforms, deep learning, high-accuracy speech, image, and text recognition systems, intelligent video analytics, molecular simulations, drug discovery, disease diagnosis, weather forecasting, big data analytics, astronomy, molecular dynamics simulation, financial modeling, robotics, factory automation, real-time language translation, online search optimizations, and personalized user recommendations, and the like.

As shown in FIG. 5, the parallel processing unit 502 includes an I/O unit 506, a front-end unit 508, a scheduler unit 510, a work distribution unit 512, a hub 514, a crossbar 516, one or more general processing cluster 518 modules, and one or more memory partition unit 520 modules. The parallel processing unit 502 may be connected to a host processor or other parallel processing unit 502 modules via one or more high-speed NVLink 522 interconnects. The parallel processing unit 502 may be connected to a host processor or other peripheral devices via an interconnect 524. The parallel processing unit 502 may also be connected to a local memory comprising a number of memory 504 devices. In an embodiment, the local memory may comprise a number of dynamic random access memory (DRAM) devices. The DRAM devices may be configured as a high-bandwidth memory (HBM) subsystem, with multiple DRAM dies stacked within each device. The memory 504 may comprise logic to configure the parallel processing unit 502 to carry out aspects of the techniques disclosed herein.

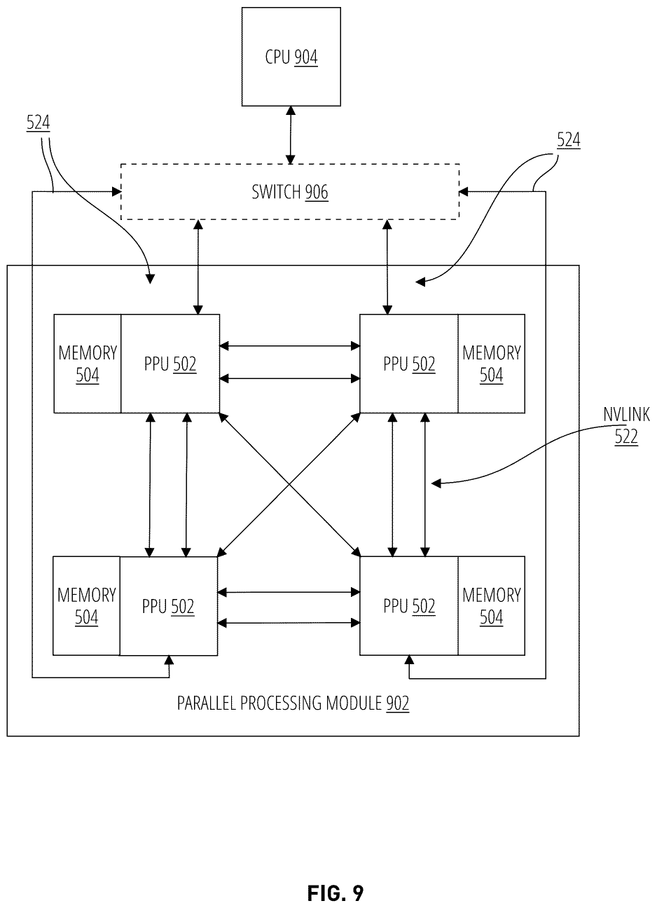

The NVLink 522 interconnect enables systems to scale and include one or more parallel processing unit 502 modules combined with one or more CPUs, supports cache coherence between the parallel processing unit 502 modules and CPUs, and CPU mastering. Data and/or commands may be transmitted by the NVLink 522 through the hub 514 to/from other units of the parallel processing unit 502 such as one or more copy engines, a video encoder, a video decoder, a power management unit, etc. (not explicitly shown). The NVLink 522 is described in more detail in conjunction with FIG. 9.

The I/O unit 506 is configured to transmit and receive communications (e.g., commands, data, etc.) from a host processor (not shown) over the interconnect 524. The I/O unit 506 may communicate with the host processor directly via the interconnect 524 or through one or more intermediate devices such as a memory bridge. In an embodiment, the I/O unit 506 may communicate with one or more other processors, such as one or more parallel processing unit 502 modules via the interconnect 524. In an embodiment, the I/O unit 506 implements a Peripheral Component Interconnect Express (PCIe) interface for communications over a PCIe bus and the interconnect 524 is a PCIe bus. In alternative embodiments, the I/O unit 506 may implement other types of well-known interfaces for communicating with external devices.

The I/O unit 506 decodes packets received via the interconnect 524. In an embodiment, the packets represent commands configured to cause the parallel processing unit 502 to perform various operations. The I/O unit 506 transmits the decoded commands to various other units of the parallel processing unit 502 as the commands may specify. For example, some commands may be transmitted to the front-end unit 508. Other commands may be transmitted to the hub 514 or other units of the parallel processing unit 502 such as one or more copy engines, a video encoder, a video decoder, a power management unit, etc. (not explicitly shown). In other words, the I/O unit 506 is configured to route communications between and among the various logical units of the parallel processing unit 502.

In an embodiment, a program executed by the host processor encodes a command stream in a buffer that provides workloads to the parallel processing unit 502 for processing. A workload may comprise several instructions and data to be processed by those instructions. The buffer is a region in a memory that is accessible (e.g., read/write) by both the host processor and the parallel processing unit 502. For example, the I/O unit 506 may be configured to access the buffer in a system memory connected to the interconnect 524 via memory requests transmitted over the interconnect 524. In an embodiment, the host processor writes the command stream to the buffer and then transmits a pointer to the start of the command stream to the parallel processing unit 502. The front-end unit 508 receives pointers to one or more command streams. The front-end unit 508 manages the one or more streams, reading commands from the streams and forwarding commands to the various units of the parallel processing unit 502.

The front-end unit 508 is coupled to a scheduler unit 510 that configures the various general processing cluster 518 modules to process tasks defined by the one or more streams. The scheduler unit 510 is configured to track state information related to the various tasks managed by the scheduler unit 510. The state may indicate which general processing cluster 518 a task is assigned to, whether the task is active or inactive, a priority level associated with the task, and so forth. The scheduler unit 510 manages the execution of a plurality of tasks on the one or more general processing cluster 518 modules.

The scheduler unit 510 is coupled to a work distribution unit 512 that is configured to dispatch tasks for execution on the general processing cluster 518 modules. The work distribution unit 512 may track a number of scheduled tasks received from the scheduler unit 510. In an embodiment, the work distribution unit 512 manages a pending task pool and an active task pool for each of the general processing cluster 518 modules. The pending task pool may comprise a number of slots (e.g., 32 slots) that contain tasks assigned to be processed by a particular general processing cluster 518. The active task pool may comprise a number of slots (e.g., 4 slots) for tasks that are actively being processed by the general processing cluster 518 modules. As a general processing cluster 518 finishes the execution of a task, that task is evicted from the active task pool for the general processing cluster 518 and one of the other tasks from the pending task pool is selected and scheduled for execution on the general processing cluster 518. If an active task has been idle on the general processing cluster 518, such as while waiting for a data dependency to be resolved, then the active task may be evicted from the general processing cluster 518 and returned to the pending task pool while another task in the pending task pool is selected and scheduled for execution on the general processing cluster 518.

The work distribution unit 512 communicates with the one or more general processing cluster 518 modules via crossbar 516. The crossbar 516 is an interconnect network that couples many of the units of the parallel processing unit 502 to other units of the parallel processing unit 502. For example, the crossbar 516 may be configured to couple the work distribution unit 512 to a particular general processing cluster 518. Although not shown explicitly, one or more other units of the parallel processing unit 502 may also be connected to the crossbar 516 via the hub 514.

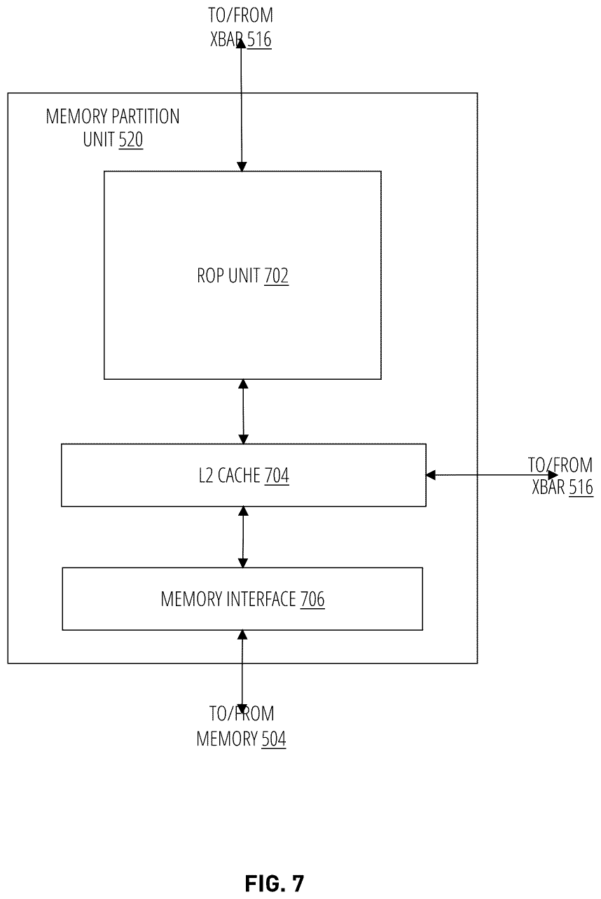

The tasks are managed by the scheduler unit 510 and dispatched to a general processing cluster 518 by the work distribution unit 512. The general processing cluster 518 is configured to process the task and generate results. The results may be consumed by other tasks within the general processing cluster 518, routed to a different general processing cluster 518 via the crossbar 516, or stored in the memory 504. The results can be written to the memory 504 via the memory partition unit 520 modules, which implement a memory interface for reading and writing data to/from the memory 504. The results can be transmitted to another parallel processing unit 502 or CPU via the NVLink 522. In an embodiment, the parallel processing unit 502 includes a number U of memory partition unit 520 modules that is equal to the number of separate and distinct memory 504 devices coupled to the parallel processing unit 502. A memory partition unit 520 will be described in more detail below in conjunction with FIG. 7.

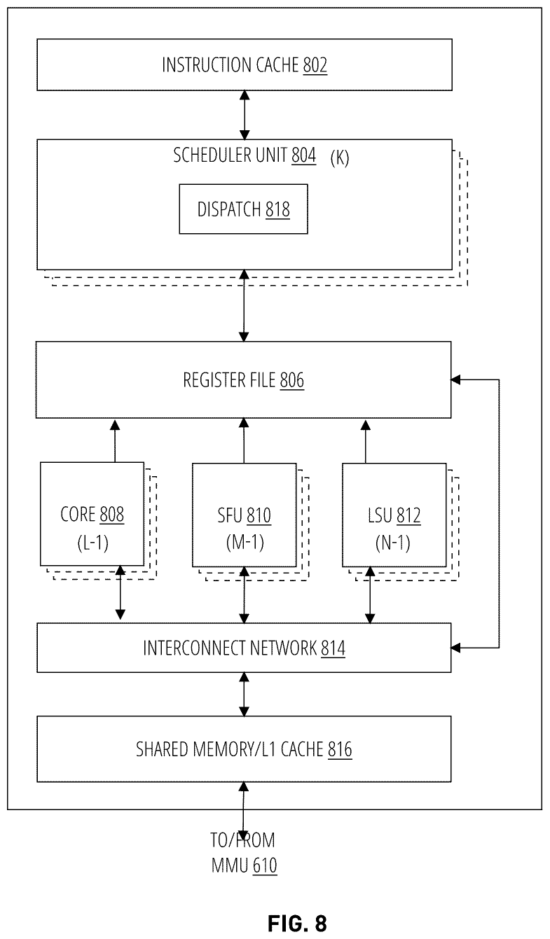

In an embodiment, a host processor executes a driver kernel that implements an application programming interface (API) that enables one or more applications executing on the host processor to schedule operations for execution on the parallel processing unit 502. In an embodiment, multiple compute applications are simultaneously executed by the parallel processing unit 502 and the parallel processing unit 502 provides isolation, quality of service (QoS), and independent address spaces for the multiple compute applications. An application may generate instructions (e.g., API calls) that cause the driver kernel to generate one or more tasks for execution by the parallel processing unit 502. The driver kernel outputs tasks to one or more streams being processed by the parallel processing unit 502. Each task may comprise one or more groups of related threads, referred to herein as a warp. In an embodiment, a warp comprises 32 related threads that may be executed in parallel. Cooperating threads may refer to a plurality of threads including instructions to perform the task and that may exchange data through shared memory. Threads and cooperating threads are described in more detail in conjunction with FIG. 8.

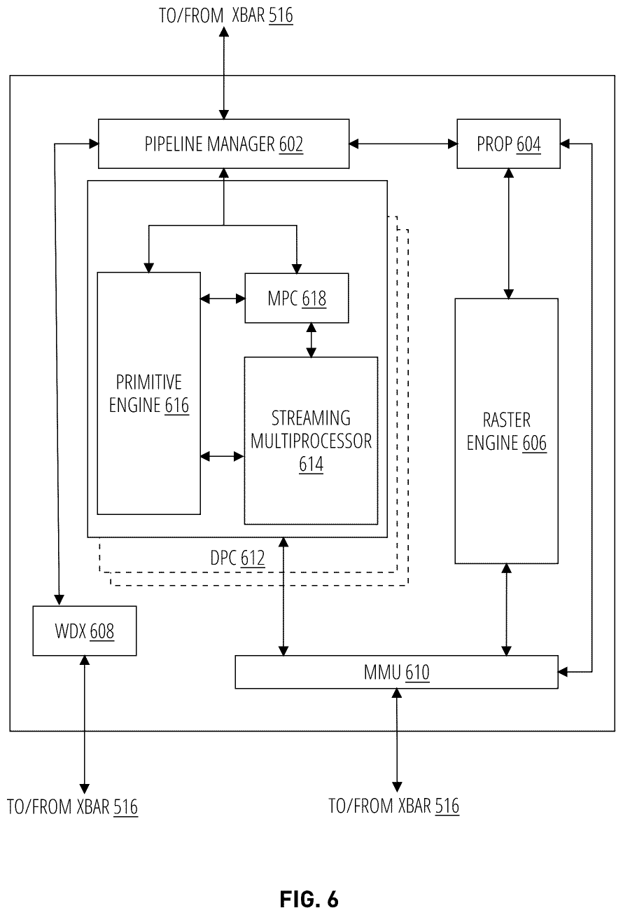

FIG. 6 depicts a general processing cluster 518 of the parallel processing unit 502 of FIG. 5, in accordance with an embodiment. As shown in FIG. 6, each general processing cluster 518 includes a number of hardware units for processing tasks. In an embodiment, each general processing cluster 518 includes a pipeline manager 602, a pre-raster operations unit 604, a raster engine 606, a work distribution crossbar 608, a memory management unit 610, and one or more data processing cluster 612. It will be appreciated that the general processing cluster 518 of FIG. 6 may include other hardware units in lieu of or in addition to the units shown in FIG. 6.

In an embodiment, the operation of the general processing cluster 518 is controlled by the pipeline manager 602. The pipeline manager 602 manages the configuration of the one or more data processing cluster 612 modules for processing tasks allocated to the general processing cluster 518. In an embodiment, the pipeline manager 602 may configure at least one of the one or more data processing cluster 612 modules to implement at least a portion of a graphics rendering pipeline. For example, a data processing cluster 612 may be configured to execute a vertex shader program on the programmable streaming multiprocessor 614. The pipeline manager 602 may also be configured to route packets received from the work distribution unit 512 to the appropriate logical units within the general processing cluster 518. For example, some packets may be routed to fixed function hardware units in the pre-raster operations unit 604 and/or raster engine 606 while other packets may be routed to the data processing cluster 612 modules for processing by the primitive engine 616 or the streaming multiprocessor 614. In an embodiment, the pipeline manager 602 may configure at least one of the one or more data processing cluster 612 modules to implement a neural network model and/or a computing pipeline.

The pre-raster operations unit 604 is configured to route data generated by the raster engine 606 and the data processing cluster 612 modules to a Raster Operations (ROP) unit, described in more detail in conjunction with FIG. 7. The pre-raster operations unit 604 may also be configured to perform optimizations for color blending, organize pixel data, perform address translations, and the like.

The raster engine 606 includes a number of fixed function hardware units configured to perform various raster operations. In an embodiment, the raster engine 606 includes a setup engine, a coarse raster engine, a culling engine, a clipping engine, a fine raster engine, and a tile coalescing engine. The setup engine receives transformed vertices and generates plane equations associated with the geometric primitive defined by the vertices. The plane equations are transmitted to the coarse raster engine to generate coverage information (e.g., an x, y coverage mask for a tile) for the primitive. The output of the coarse raster engine is transmitted to the culling engine where fragments associated with the primitive that fail a z-test are culled, and transmitted to a clipping engine where fragments lying outside a viewing frustum are clipped. Those fragments that survive clipping and culling may be passed to the fine raster engine to generate attributes for the pixel fragments based on the plane equations generated by the setup engine. The output of the raster engine 606 comprises fragments to be processed, for example, by a fragment shader implemented within a data processing cluster 612.

Each data processing cluster 612 included in the general processing cluster 518 includes an M-pipe controller 618, a primitive engine 616, and one or more streaming multiprocessor 614 modules. The M-pipe controller 618 controls the operation of the data processing cluster 612, routing packets received from the pipeline manager 602 to the appropriate units in the data processing cluster 612. For example, packets associated with a vertex may be routed to the primitive engine 616, which is configured to fetch vertex attributes associated with the vertex from the memory 504. In contrast, packets associated with a shader program may be transmitted to the streaming multiprocessor 614.

The streaming multiprocessor 614 comprises a programmable streaming processor that is configured to process tasks represented by a number of threads. Each streaming multiprocessor 614 is multi-threaded and configured to execute a plurality of threads (e.g., 32 threads) from a particular group of threads concurrently. In an embodiment, the streaming multiprocessor 614 implements a Single-Instruction, Multiple-Data (SIMD) architecture where each thread in a group of threads (e.g., a warp) is configured to process a different set of data based on the same set of instructions. All threads in the group of threads execute the same instructions. In another embodiment, the streaming multiprocessor 614 implements a Single-Instruction, Multiple Thread (SIMT) architecture where each thread in a group of threads is configured to process a different set of data based on the same set of instructions, but where individual threads in the group of threads are allowed to diverge during execution. In an embodiment, a program counter, call stack, and execution state is maintained for each warp, enabling concurrency between warps and serial execution within warps when threads within the warp diverge. In another embodiment, a program counter, call stack, and execution state is maintained for each individual thread, enabling equal concurrency between all threads, within and between warps. When execution state is maintained for each individual thread, threads executing the same instructions may be converged and executed in parallel for maximum efficiency. The streaming multiprocessor 614 will be described in more detail below in conjunction with FIG. 8.

The memory management unit 610 provides an interface between the general processing cluster 518 and the memory partition unit 520. The memory management unit 610 may provide translation of virtual addresses into physical addresses, memory protection, and arbitration of memory requests. In an embodiment, the memory management unit 610 provides one or more translation lookaside buffers (TLBs) for performing translation of virtual addresses into physical addresses in the memory 504.

FIG. 7 depicts a memory partition unit 520 of the parallel processing unit 502 of FIG. 5, in accordance with an embodiment. As shown in FIG. 7, the memory partition unit 520 includes a raster operations unit 702, a level two cache 704, and a memory interface 706. The memory interface 706 is coupled to the memory 504. Memory interface 706 may implement 32, 64, 128, 1024-bit data buses, or the like, for high-speed data transfer. In an embodiment, the parallel processing unit 502 incorporates U memory interface 706 modules, one memory interface 706 per pair of memory partition unit 520 modules, where each pair of memory partition unit 520 modules is connected to a corresponding memory 504 device. For example, parallel processing unit 502 may be connected to up to Y memory 504 devices, such as high bandwidth memory stacks or graphics double-data-rate, version 5, synchronous dynamic random access memory, or other types of persistent storage.

In an embodiment, the memory interface 706 implements an HBM2 memory interface and Y equals half U. In an embodiment, the HBM2 memory stacks are located on the same physical package as the parallel processing unit 502, providing substantial power and area savings compared with conventional GDDR5 SDRAM systems. In an embodiment, each HBM2 stack includes four memory dies and Y equals 4, with HBM2 stack including two 128-bit channels per die for a total of 8 channels and a data bus width of 1024 bits.

In an embodiment, the memory 504 supports Single-Error Correcting Double-Error Detecting (SECDED) Error Correction Code (ECC) to protect data. ECC provides higher reliability for compute applications that are sensitive to data corruption. Reliability is especially important in large-scale cluster computing environments where parallel processing unit 502 modules process very large datasets and/or run applications for extended periods.

In an embodiment, the parallel processing unit 502 implements a multi-level memory hierarchy. In an embodiment, the memory partition unit 520 supports a unified memory to provide a single unified virtual address space for CPU and parallel processing unit 502 memory, enabling data sharing between virtual memory systems. In an embodiment the frequency of accesses by a parallel processing unit 502 to memory located on other processors is traced to ensure that memory pages are moved to the physical memory of the parallel processing unit 502 that is accessing the pages more frequently. In an embodiment, the NVLink 522 supports address translation services allowing the parallel processing unit 502 to directly access a CPU's page tables and providing full access to CPU memory by the parallel processing unit 502.

In an embodiment, copy engines transfer data between multiple parallel processing unit 502 modules or between parallel processing unit 502 modules and CPUs. The copy engines can generate page faults for addresses that are not mapped into the page tables. The memory partition unit 520 can then service the page faults, mapping the addresses into the page table, after which the copy engine can perform the transfer. In a conventional system, memory is pinned (e.g., non-pageable) for multiple copy engine operations between multiple processors, substantially reducing the available memory. With hardware page faulting, addresses can be passed to the copy engines without worrying if the memory pages are resident, and the copy process is transparent.

Data from the memory 504 or other system memory may be fetched by the memory partition unit 520 and stored in the level two cache 704, which is located on-chip and is shared between the various general processing cluster 518 modules. As shown, each memory partition unit 520 includes a portion of the level two cache 704 associated with a corresponding memory 504 device. Lower level caches may then be implemented in various units within the general processing cluster 518 modules. For example, each of the streaming multiprocessor 614 modules may implement an L1 cache. The L1 cache is private memory that is dedicated to a particular streaming multiprocessor 614. Data from the level two cache 704 may be fetched and stored in each of the L1 caches for processing in the functional units of the streaming multiprocessor 614 modules. The level two cache 704 is coupled to the memory interface 706 and the crossbar 516.

The raster operations unit 702 performs graphics raster operations related to pixel color, such as color compression, pixel blending, and the like. The raster operations unit 702 also implements depth testing in conjunction with the raster engine 606, receiving a depth for a sample location associated with a pixel fragment from the culling engine of the raster engine 606. The depth is tested against a corresponding depth in a depth buffer for a sample location associated with the fragment. If the fragment passes the depth test for the sample location, then the raster operations unit 702 updates the depth buffer and transmits a result of the depth test to the raster engine 606. It will be appreciated that the number of partition memory partition unit 520 modules may be different than the number of general processing cluster 518 modules and, therefore, each raster operations unit 702 may be coupled to each of the general processing cluster 518 modules. The raster operations unit 702 tracks packets received from the different general processing cluster 518 modules and determines which general processing cluster 1 that a result generated by the raster operations unit 702 is routed to through the crossbar 516. Although the raster operations unit 702 is included within the memory partition unit 520 in FIG. 7, in other embodiment, the raster operations unit 702 may be outside of the memory partition unit 520. For example, the raster operations unit 702 may reside in the general processing cluster 518 or another unit.

FIG. 8 illustrates the streaming multiprocessor 614 of FIG. 6, in accordance with an embodiment. As shown in FIG. 8, the streaming multiprocessor 614 includes an instruction cache 802, one or more scheduler unit 804 modules (e.g., such as scheduler unit 510), a register file 806, one or more processing core 808 modules, one or more special function unit 810 modules, one or more load/store unit 812 modules, an interconnect network 814, and a shared memory/L1 cache 816.

As described above, the work distribution unit 512 dispatches tasks for execution on the general processing cluster 518 modules of the parallel processing unit 502. The tasks are allocated to a particular data processing cluster 612 within a general processing cluster 518 and, if the task is associated with a shader program, the task may be allocated to a streaming multiprocessor 614. The scheduler unit 510 receives the tasks from the work distribution unit 512 and manages instruction scheduling for one or more thread blocks assigned to the streaming multiprocessor 614. The scheduler unit 804 schedules thread blocks for execution as warps of parallel threads, where each thread block is allocated at least one warp. In an embodiment, each warp executes 32 threads. The scheduler unit 804 may manage a plurality of different thread blocks, allocating the warps to the different thread blocks and then dispatching instructions from the plurality of different cooperative groups to the various functional units (e.g., core 808 modules, special function unit 810 modules, and load/store unit 812 modules) during each clock cycle.

Cooperative Groups is a programming model for organizing groups of communicating threads that allows developers to express the granularity at which threads are communicating, enabling the expression of richer, more efficient parallel decompositions. Cooperative launch APIs support synchronization amongst thread blocks for the execution of parallel algorithms. Conventional programming models provide a single, simple construct for synchronizing cooperating threads: a barrier across all threads of a thread block (e.g., the syncthreads ( ) function). However, programmers would often like to define groups of threads at smaller than thread block granularities and synchronize within the defined groups to enable greater performance, design flexibility, and software reuse in the form of collective group-wide function interfaces.

Cooperative Groups enables programmers to define groups of threads explicitly at sub-block (e.g., as small as a single thread) and multi-block granularities, and to perform collective operations such as synchronization on the threads in a cooperative group. The programming model supports clean composition across software boundaries, so that libraries and utility functions can synchronize safely within their local context without having to make assumptions about convergence. Cooperative Groups primitives enable new patterns of cooperative parallelism, including producer-consumer parallelism, opportunistic parallelism, and global synchronization across an entire grid of thread blocks.

A dispatch 818 unit is configured within the scheduler unit 804 to transmit instructions to one or more of the functional units. In one embodiment, the scheduler unit 804 includes two dispatch 818 units that enable two different instructions from the same warp to be dispatched during each clock cycle. In alternative embodiments, each scheduler unit 804 may include a single dispatch 818 unit or additional dispatch 818 units.

Each streaming multiprocessor 614 includes a register file 806 that provides a set of registers for the functional units of the streaming multiprocessor 614. In an embodiment, the register file 806 is divided between each of the functional units such that each functional unit is allocated a dedicated portion of the register file 806. In another embodiment, the register file 806 is divided between the different warps being executed by the streaming multiprocessor 614. The register file 806 provides temporary storage for operands connected to the data paths of the functional units.

Each streaming multiprocessor 614 comprises L processing core 808 modules. In an embodiment, the streaming multiprocessor 614 includes a large number (e.g., 128, etc.) of distinct processing core 808 modules. Each core 808 may include a fully-pipelined, single-precision, double-precision, and/or mixed precision processing unit that includes a floating point arithmetic logic unit and an integer arithmetic logic unit. In an embodiment, the floating point arithmetic logic units implement the IEEE 754-2008 standard for floating point arithmetic. In an embodiment, the core 808 modules include 64 single-precision (32-bit) floating point cores, 64 integer cores, 32 double-precision (64-bit) floating point cores, and 8 tensor cores.

Tensor cores configured to perform matrix operations, and, in an embodiment, one or more tensor cores are included in the core 808 modules. In particular, the tensor cores are configured to perform deep learning matrix arithmetic, such as convolution operations for neural network training and inferencing. In an embodiment, each tensor core operates on a 4×4 matrix and performs a matrix multiply and accumulate operation D=A′B+C, where A, B, C, and D are 4×4 matrices.

In an embodiment, the matrix multiply inputs A and B are 16-bit floating point matrices, while the accumulation matrices C and D may be 16-bit floating point or 32-bit floating point matrices. Tensor Cores operate on 16-bit floating point input data with 32-bit floating point accumulation. The 16-bit floating point multiply requires 64 operations and results in a full precision product that is then accumulated using 32-bit floating point addition with the other intermediate products for a 4×4×4 matrix multiply. In practice, Tensor Cores are used to perform much larger two-dimensional or higher dimensional matrix operations, built up from these smaller elements. An API, such as CUDA 9 C++ API, exposes specialized matrix load, matrix multiply and accumulate, and matrix store operations to efficiently use Tensor Cores from a CUDA-C++ program. At the CUDA level, the warp-level interface assumes 16×16 size matrices spanning all 32 threads of the warp.

Each streaming multiprocessor 614 also comprises M special function unit 810 modules that perform special functions (e.g., attribute evaluation, reciprocal square root, and the like). In an embodiment, the special function unit 810 modules may include a tree traversal unit configured to traverse a hierarchical tree data structure. In an embodiment, the special function unit 810 modules may include texture unit configured to perform texture map filtering operations. In an embodiment, the texture units are configured to load texture maps (e.g., a 2D array of texels) from the memory 504 and sample the texture maps to produce sampled texture values for use in shader programs executed by the streaming multiprocessor 614. In an embodiment, the texture maps are stored in the shared memory/L1 cache 816. The texture units implement texture operations such as filtering operations using mip-maps (e.g., texture maps of varying levels of detail). In an embodiment, each streaming multiprocessor 614 includes two texture units.

Each streaming multiprocessor 614 also comprises N load/store unit 812 modules that implement load and store operations between the shared memory/L1 cache 816 and the register file 806. Each streaming multiprocessor 614 includes an interconnect network 814 that connects each of the functional units to the register file 806 and the load/store unit 812 to the register file 806 and shared memory/L1 cache 816. In an embodiment, the interconnect network 814 is a crossbar that can be configured to connect any of the functional units to any of the registers in the register file 806 and connect the load/store unit 812 modules to the register file 806 and memory locations in shared memory/L1 cache 816.

The shared memory/L1 cache 816 is an array of on-chip memory that allows for data storage and communication between the streaming multiprocessor 614 and the primitive engine 616 and between threads in the streaming multiprocessor 614. In an embodiment, the shared memory/L1 cache 816 comprises 128 KB of storage capacity and is in the path from the streaming multiprocessor 614 to the memory partition unit 520. The shared memory/L1 cache 816 can be used to cache reads and writes. One or more of the shared memory/L1 cache 816, level two cache 704, and memory 504 are backing stores.

Combining data cache and shared memory functionality into a single memory block provides the best overall performance for both types of memory accesses. The capacity is usable as a cache by programs that do not use shared memory. For example, if shared memory is configured to use half of the capacity, texture and load/store operations can use the remaining capacity. Integration within the shared memory/L1 cache 816 enables the shared memory/L1 cache 816 to function as a high-throughput conduit for streaming data while simultaneously providing high-bandwidth and low-latency access to frequently reused data.

When configured for general purpose parallel computation, a simpler configuration can be used compared with graphics processing. Specifically, the fixed function graphics processing units shown in FIG. 5, are bypassed, creating a much simpler programming model. In the general purpose parallel computation configuration, the work distribution unit 512 assigns and distributes blocks of threads directly to the data processing cluster 612 modules. The threads in a block execute the same program, using a unique thread ID in the calculation to ensure each thread generates unique results, using the streaming multiprocessor 614 to execute the program and perform calculations, shared memory/L1 cache 816 to communicate between threads, and the load/store unit 812 to read and write global memory through the shared memory/L1 cache 816 and the memory partition unit 520. When configured for general purpose parallel computation, the streaming multiprocessor 614 can also write commands that the scheduler unit 510 can use to launch new work on the data processing cluster 612 modules.

The parallel processing unit 502 may be included in a desktop computer, a laptop computer, a tablet computer, servers, supercomputers, a smart-phone (e.g., a wireless, hand-held device), personal digital assistant (PDA), a digital camera, a vehicle, a head mounted display, a hand-held electronic device, and the like. In an embodiment, the parallel processing unit 502 is embodied on a single semiconductor substrate. In another embodiment, the parallel processing unit 502 is included in a system-on-a-chip (SoC) along with one or more other devices such as additional parallel processing unit 502 modules, the memory 504, a reduced instruction set computer (RISC) CPU, a memory management unit (MMU), a digital-to-analog converter (DAC), and the like.

In an embodiment, the parallel processing unit 502 may be included on a graphics card that includes one or more memory devices. The graphics card may be configured to interface with a PCIe slot on a motherboard of a desktop computer. In yet another embodiment, the parallel processing unit 502 may be an integrated graphics processing unit (iGPU) or parallel processor included in the chipset of the motherboard.

Systems with multiple GPUs and CPUs are used in a variety of industries as developers expose and leverage more parallelism in applications such as artificial intelligence computing. High-performance GPU-accelerated systems with tens to many thousands of compute nodes are deployed in data centers, research facilities, and supercomputers to solve ever larger problems. As the number of processing devices within the high-performance systems increases, the communication and data transfer mechanisms need to scale to support the increased bandwidth.

FIG. 9 is a conceptual diagram of a processing system implemented using the parallel processing unit 502 of FIG. 5, in accordance with an embodiment. The processing system includes a central processing unit 904, a switch 906, and multiple parallel processing unit 502 modules each and respective memory 504 modules. The switch 906 is depicted with dashed lines, indicating that it is optional in some embodiments.

The NVLink 522 provides high-speed communication links between each of the parallel processing unit 502 modules. Although a particular number of NVLink 522 and interconnect 524 connections are illustrated in FIG. 9, the number of connections to each parallel processing unit 502 and the central processing unit 904 may vary. The switch 906 interfaces between the interconnect 524 and the central processing unit 904. The parallel processing unit 502 modules, memory 504 modules, and NVLink 522 connections may be situated on a single semiconductor platform to form a parallel processing module 902. In an embodiment, the switch 906 supports two or more protocols to interface between various different connections and/or links.

In another embodiment (not shown), the NVLink 522 provides one or more high-speed communication links between each of the parallel processing unit modules (parallel processing unit 502, parallel processing unit 502, parallel processing unit 502, and parallel processing unit 502) and the central processing unit 904 and the switch 906 (when present) interfaces between the interconnect 524 and each of the parallel processing unit modules. The parallel processing unit modules, memory 504 modules, and interconnect 524 may be situated on a single semiconductor platform to form a parallel processing module 902. In yet another embodiment (not shown), the interconnect 524 provides one or more communication links between each of the parallel processing unit modules and the central processing unit 904 and the switch 906 interfaces between each of the parallel processing unit modules using the NVLink 522 to provide one or more high-speed communication links between the parallel processing unit modules. In another embodiment (not shown), the NVLink 522 provides one or more high-speed communication links between the parallel processing unit modules and the central processing unit 904 through the switch 906. In yet another embodiment (not shown), the interconnect 524 provides one or more communication links between each of the parallel processing unit modules directly. One or more of the NVLink 522 high-speed communication links may be implemented as a physical NVLink interconnect or either an on-chip or on-die interconnect using the same protocol as the NVLink 522.

In the context of the present description, a single semiconductor platform may refer to a sole unitary semiconductor-based integrated circuit fabricated on a die or chip. It should be noted that the term single semiconductor platform may also refer to multi-chip modules with increased connectivity which simulate on-chip operation and make substantial improvements over utilizing a conventional bus implementation. Of course, the various circuits or devices may also be situated separately or in various combinations of semiconductor platforms per the desires of the user. Alternately, the parallel processing module 902 may be implemented as a circuit board substrate and each of the parallel processing unit modules and/or memory 504 modules may be packaged devices. In an embodiment, the central processing unit 904, switch 906, and the parallel processing module 902 are situated on a single semiconductor platform.

In an embodiment, each parallel processing unit module includes six NVLink 522 interfaces (as shown in FIG. 9, five NVLink 522 interfaces are included for each parallel processing unit module). The NVLink 522 may be operated exclusively for PPU-to-PPU communication as shown in FIG. 9, or some combination of PPU-to-PPU and PPU-to-CPU, when the central processing unit 904 also includes one or more NVLink 522 interfaces.

In an embodiment, the NVLink 522 allows direct load/store/atomic access from the central processing unit 904 to each parallel processing unit module's memory 504. In an embodiment, the NVLink 522 supports coherency operations, allowing data read from the memory 504 modules to be stored in the cache hierarchy of the central processing unit 904, reducing cache access latency for the central processing unit 904. In an embodiment, the NVLink 522 includes support for Address Translation Services (ATS), enabling the parallel processing unit module to directly access page tables within the central processing unit 904. One or more of the NVLink 522 may also be configured to operate in a low-power mode.

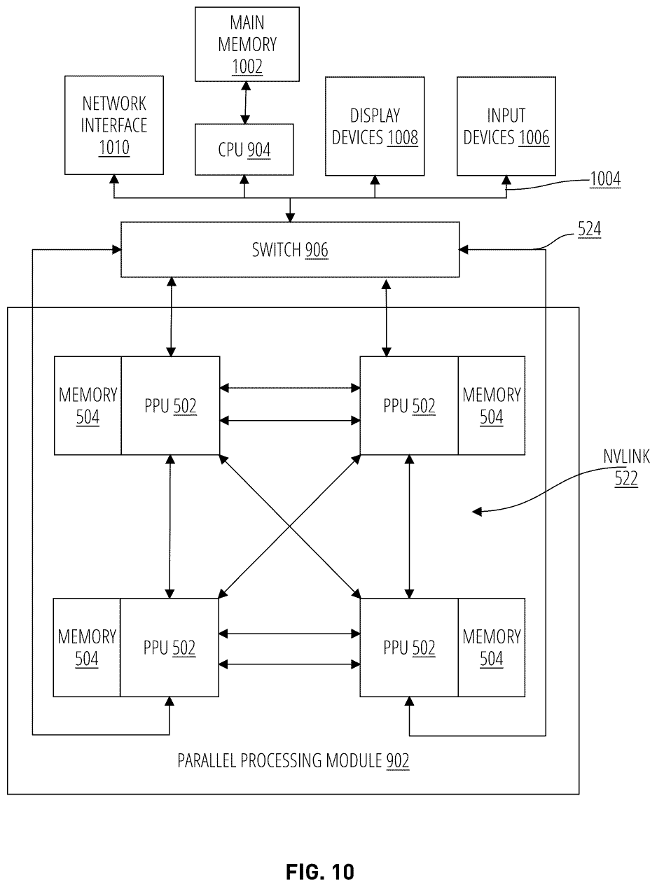

FIG. 10 depicts an exemplary processing system in which the various architecture and/or functionality of the various previous embodiments may be implemented. As shown, an exemplary processing system is provided including at least one central processing unit 904 that is connected to a communications bus 1004. The communication communications bus 1004 may be implemented using any suitable protocol, such as PCI (Peripheral Component Interconnect), PCI-Express, AGP (Accelerated Graphics Port), HyperTransport, or any other bus or point-to-point communication protocol(s). The exemplary processing system also includes a main memory 1002. Control logic (software) and data are stored in the main memory 1002 which may take the form of random access memory (RAM). For simplicity of illustration, the main memory 1002 may be understood to comprise other forms of bulk memory, including non-volatile memory technologies.

The exemplary processing system also includes input devices 1006, the parallel processing module 902, and display devices 1008, e.g. a conventional CRT (cathode ray tube), LCD (liquid crystal display), LED (light emitting diode), plasma display or the like. User input may be received from the input devices 1006, e.g., keyboard, mouse, touchpad, microphone, and the like. Each of the foregoing modules and/or devices may even be situated on a single semiconductor platform to form the exemplary processing system. Alternately, the various modules may also be situated separately or in various combinations of semiconductor platforms per the desires of the user.

Further, the exemplary processing system may be coupled to a network (e.g., a telecommunications network, local area network (LAN), wireless network, wide area network (WAN) such as the Internet, peer-to-peer network, cable network, or the like) through a network interface 1010 for communication purposes.

The exemplary processing system may also include a secondary storage (not shown). The secondary storage includes, for example, a hard disk drive and/or a removable storage drive, representing a floppy disk drive, a magnetic tape drive, a compact disk drive, digital versatile disk (DVD) drive, recording device, universal serial bus (USB) flash memory. The removable storage drive reads from and/or writes to a removable storage unit in a well-known manner.

Computer programs, or computer control logic algorithms, may be stored in the main memory 1002 and/or the secondary storage. Such computer programs, when executed, enable the exemplary processing system to perform various functions. The main memory 1002, the storage, and/or any other storage are possible examples of computer-readable media (volatile and/or non-volatile, depending on the implementation).

The architecture and/or functionality of the various previous figures may be implemented in the context of a general computer system, a circuit board system, a game console system dedicated for entertainment purposes, an application-specific system, and/or any other desired system. For example, the exemplary processing system may take the form of a desktop computer, a laptop computer, a tablet computer, servers, supercomputers, a smart-phone (e.g., a wireless, hand-held device), personal digital assistant (PDA), a digital camera, a vehicle, a head mounted display, a hand-held electronic device, a mobile phone device, a television, workstation, game consoles, embedded system, and/or any other type of logic.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

### LISTING OF DRAWING ELEMENTS

302 model
304 transformer block
306 classifier network
402 fragment vocabulary
404 molecular structures
502 parallel processing unit
504 memory
506 I/O unit
508 front-end unit
510 scheduler unit
512 work distribution unit
514 hub
516 crossbar
518 general processing cluster
520 memory partition unit
522 NVLink
524 interconnect
602 pipeline manager
604 pre-raster operations unit
606 raster engine
608 work distribution crossbar
610 memory management unit
612 data processing cluster
614 streaming multiprocessor
616 primitive engine
618 M-pipe controller
702 raster operations unit
704 level two cache
706 memory interface
802 instruction cache
804 scheduler unit
806 register file
808 core
810 special function unit
812 load/store unit
814 interconnect network
816 shared memory/L1 cache
818 dispatch
902 parallel processing module
904 central processing unit
906 switch
1002 main memory
1004 communications bus
1006 input devices
1008 display devices
1010 network interface

Artificial neural networks are commonly deployed as components of data centers, desktop and laptop computers, mobile phones, and many other devices, and are useful in a wide range of practical applications. For example, artificial neural networks are commonly utilized as the basis of artificial intelligence (AI) systems. Some examples of systems that may incorporate artificial neural networks are high-performance computers and rack systems, vehicles, and robots, to name a few examples.

An artificial neural network comprises, at a high level, a structuring of connected nodes organized into layers, with feed-forward and/or feedback signaling paths configured between the nodes and layers. Signals on the connections between nodes and layers are selectively activated and weighted by amounts determined by the neural network's configuration during training. Practical neural network topologies comprise millions or billions of uniquely configured weights, activation values, and interconnections, as well other settings such as biases and thresholds, encoded as physical electronic states in machine memory. A neural network's structure and layer configurations dictate physical memory access behaviors and electronic activation pathways through hardware computational circuits when executing AI workloads.

Different neural network structures and layer configurations result in different utilizations of computer hardware circuits and memory. Neural networks encoded in computer instructions are highly engineered memory structures that dictate the activation of particular hardware circuits and flow of electrical signals to and through specific memory addresses and hardware circuits of a computer system. Execution of a neural network structure causes specific current flows and energy consumption by specific hardware logic gates in the computer system's processing cores. The specific state changes and hardware component activations induced in a computer system by execution of a particular neural network structure are inseparable from that neural network structure.

During training or inference, any execution pass through neurons and layers of an artificial neural network structure loads network parameters over memory buses, activates specific hardware components (e.g., of GPU tensor cores or CPU ALUs) and triggers the operation of other specific circuits. The structure and configuration of the neural network dictates the physical computation pathways at the microarchitectural level.

Neural network structures are machine-implemented computational architecture that operate analogously to specific electronic circuits. Although a neural network structure may be described to some extent using mathematical formalisms, it exists as a physical, engineered artifact. A computer system comprising an artificial neural network layer structure constitutes “a particular machine,” not a general-purpose computer.

Various functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on. “Logic” refers to machine memory circuits and non-transitory machine readable media configured with machine-executable instructions (software and firmware), and/or circuitry (hardware) which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory, and firmware are examples of logic. Logic specifically excludes pure signals or software per se (however does not exclude non-transitory machine memories comprising software and thereby forming statutory configurations of matter). Logic symbols in the drawings should be understood to have their ordinary interpretation in the art in terms of functionality and various structures that may be utilized for their implementation, unless otherwise indicated.

Within this disclosure, different entities (which may variously be referred to as “units,” “circuits,” other components, etc.) may be described or claimed as “configured” to perform one or more tasks or operations. This formulation-[entity] configured to [perform one or more tasks]—is used herein to refer to structure (i.e., something physical, such as an electronic circuit). More specifically, this formulation is used to indicate that this structure is arranged to perform the one or more tasks during operation. A structure can be said to be “configured to” perform some task even if the structure is not currently being operated. A “credit distribution circuit configured to distribute credits to a plurality of processor cores” is intended to cover, for example, an integrated circuit that has circuitry that performs this function during operation, even if the integrated circuit in question is not currently being used (e.g., a power supply is not connected to it). Thus, an entity described or recited as “configured to” perform some task refers to something physical, such as a device, circuit, memory storing program instructions executable to implement the task, etc. This phrase is not used herein to refer to something intangible.

The term “configured to” is not intended to mean “configurable to.” An unprogrammed FPGA, for example, would not be considered to be “configured to” perform some specific function, although it may be “configurable to” perform that function after programming.

Reciting in the appended claims that a structure is “configured to” perform one or more tasks is expressly intended not to invoke 35 U.S.C. § 112 (f) for that claim element. Accordingly, claims in this application that do not otherwise include the “means for” [performing a function] construct should not be interpreted under 35 U.S.C § 112 (f).

As used herein, the term “based on” is used to describe one or more factors that affect a determination. This term does not foreclose the possibility that additional factors may affect the determination. That is, a determination may be solely based on specified factors or based on the specified factors as well as other, unspecified factors. Consider the phrase “determine A based on B.” This phrase specifies that B is a factor that is used to determine A or that affects the determination of A. This phrase does not foreclose that the determination of A may also be based on some other factor, such as C. This phrase is also intended to cover an embodiment in which A is determined based solely on B. As used herein, the phrase “based on” is synonymous with the phrase “based at least in part on.”

As used herein, the phrase “in response to” describes one or more factors that trigger an effect. This phrase does not foreclose the possibility that additional factors may affect or otherwise trigger the effect. That is, an effect may be solely in response to those factors, or may be in response to the specified factors as well as other, unspecified factors. Consider the phrase “perform A in response to B.” This phrase specifies that B is a factor that triggers the performance of A. This phrase does not foreclose that performing A may also be in response to some other factor, such as C. This phrase is also intended to cover an embodiment in which A is performed solely in response to B.

As used herein, the terms “first,” “second,” etc. are used as labels for nouns that they precede, and do not imply any type of ordering (e.g., spatial, temporal, logical, etc.), unless stated otherwise. For example, in a register file having eight registers, the terms “first register” and “second register” can be used to refer to any two of the eight registers, and not, for example, just logical registers 0 and 1.

When used in the claims, the term “or” is used as an inclusive or and not as an exclusive or. For example, the phrase “at least one of x, y, or z” means any one of x, y, and z, as well as any combination thereof.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B.

Although the terms “step” and/or “block” may be used herein to connote different elements of methods employed, the terms should not be interpreted as implying any particular order among or between various steps herein disclosed unless and except when the order of individual steps is explicitly described.

Having thus described illustrative embodiments in detail, it will be apparent that modifications and variations are possible without departing from the scope of the disclosure as claimed. The scope of inventive subject matter is not limited to the depicted embodiments but is rather set forth in the following Claims.

What is claimed is:

1. A process comprising:
operating a masked discrete diffusion model with fragment remasking to generates molecular sequences; and
conditioning the operation of the masked discrete diffusion model with molecular context guidance.

2. The process of claim 1, wherein the molecular sequences are SAFE sequences.

3. The process of claim 1, wherein the masked discrete diffusion model is structured as a masked diffusion language model.

4. The process of claim 3, wherein the masked diffusion language model comprises a BERT structure.

5. The process of claim 1, further comprising:
training the masked discrete diffusion model with Negative Evidence Lower Bound loss.

6. The process of claim 1, wherein generation of the molecular sequences comprises:
fragment candidate generation;
fragment candidate scoring;
fragment attaching; and
fragment remasking.

7. The process of claim 1, wherein the molecular context guidance comprises:
extrapolation between predictions of two denoising operations.

8. The process of claim 7, wherein the two denoising operations are implemented by a same denoising network configured with a first input and a corrupted version of the first input.

9. The process of claim 1, wherein the fragment remasking comprises:
sampling varying lengths of fragment mask chunks from a predefined distribution.

10. A non-volatile machine-readable media comprising instructions that, when applied to one or more data processors of a computer system, configure the computer system to:
operate a masked discrete diffusion model with fragment remasking to generates molecular sequences; and
condition the operation of the masked discrete diffusion model with molecular context guidance.

11. The non-volatile machine-readable media of claim 10, wherein the molecular sequences are SAFE sequences.

12. The non-volatile machine-readable media of claim 10, wherein the masked discrete diffusion model is structured as a masked diffusion language model.

13. The non-volatile machine-readable media of claim 12, wherein the masked diffusion language model comprises a BERT structure.

14. The non-volatile machine-readable media of claim 10, further comprising instructions that, when applied to the one or more data processors of the computer system, configure the computer system to:
train the masked discrete diffusion model with Negative Evidence Lower Bound loss.

15. The non-volatile machine-readable media of claim 10, wherein generation of the molecular sequences comprises:
fragment candidate generation;
fragment candidate scoring;
fragment attaching; and
fragment remasking.

16. The non-volatile machine-readable media of claim 10, wherein the molecular context guidance comprises:
extrapolation between predictions of two denoising operations.

17. The non-volatile machine-readable media of claim 16, wherein the two denoising operations are implemented by a same denoising network configured with a first input and a corrupted version of the first input.

18. The non-volatile machine-readable media of claim 10, wherein the fragment remasking comprises:
sampling varying lengths of fragment mask chunks from a predefined distribution.

19. A computer system comprising:
at least one graphics processing unit; and
logic configured to operate the at least one graphics processing unit to generate molecular sequences by:
generating molecular fragment candidates;
masking the molecular fragment candidates;
scoring the molecular fragment candidates;
attaching the molecular fragment candidates to a molecule;
operating a masked discrete diffusion model on the molecule to generates the molecular sequences, where operation of the masked discrete diffusion model is conditioned with molecular context guidance.

20. The computer system of claim 19, wherein the masked discrete diffusion model is structured as a masked diffusion language model.
